(19) United States
(12) Patent Application Publication
(10) Pub. No.: US 2008/0146695 A1
(43) Pub. Date: Jun. 19, 2008

(54) Molding Compositions

(76) Inventors: Hartmut Nefzger, Pulheim (DE); Erika Bauer, Juchen (DE); Michael Ludewig, Leverkusen (DE); Matthias Schaub, Linsengericht (DE); Klaus-Dieter Nehren, Dormagen (DE); Michael Freckmann, Koln (DE); Holger Urbas, Krefeld (DE)

Correspondence Address: BAYER MATERIAL SCIENCE LLC, 100 BAYER ROAD, PITTSBURGH, PA, 15205, US

(21) Appl. No.: 11/986,146

(22) Filed: Nov. 20, 2007

(30) Foreign Application Priority Data
Nov. 25, 2006 (DE) 10 2006 055739.5

(51) Int. Cl. A61K 6/10; C08G 77/00

(52) U.S. Cl. 523/109; 528/14; 528/28

(57) ABSTRACT

The present invention relates to molding compositions based on silane-terminated polyether derivatives, to a process for their preparation and to their use.

### CROSS-REFERENCE TO RELATED APPLICATIONS

The present application claims the right of priority under 35 U.S.C. §119 (a)-(d) of German Patent Application Number 10 2006 055739.5, filed Nov. 25, 2006.

### BACKGROUND OF THE INVENTION

The present invention relates to molding compositions based on polyether derivatives, to a process for their preparation and to their use.

Molding compositions based on polyether derivatives, which are used in the dental sector, have been known for a long time. According to the prior art, pastes are used whose components include, for example, polyether polyols, polyisocyanates and aminosiloxanes as well as, in addition, fillers and further auxiliary substances.

Crosslinking of the compositions takes place, for example, by the hydrolysis of alkoxysilane groups by ambient moisture or moisture added specifically, followed by crosslinking to form siloxane groupings.

The demands made of dental molding compositions are very high. EP-A 0 269 819 mentions, inter alia, a pleasant taste and odor, an aesthetically pleasing appearance, good storage stability, good handling ability, precision of the moldings, usable curing characteristics, and molded bodies that are dimensionally stable under ambient conditions. Furthermore, such compositions must not contain any irritating or toxic constituents. Cured compositions must, of course, have good deformation behavior under pressure and, where possible, must not exhibit hysteresis under tensile load. In addition, it must be possible to produce them in an economically advantageous manner.

Earlier solutions to that problem involve, for example, alginate molding compositions, which have the disadvantage of comparatively great shrinkage. Polysulfide molding compositions are dark in color and in addition also contain lead or copper compounds as catalysts. Polyether molding compositions contain ethyleneimine crosslinkers. Polysiloxane molding compositions occasionally give faulty impressions owing to the moisture in the oral cavity.

The closest prior art is disclosed in EP-A 1 245 601 and EP-A 0 269 819.

According to EP-A 1 245 601, first the preparation of a NCO prepolymer from a polyol and an aliphatic, cycloaliphatic or aromatic polyisocyanate is described, characterized in that there is no metal catalysis. This is also true of the second stage, in which the NCO prepolymer is reacted with secondary amine-terminated aminoalkylalkoxysilane.

Of course, that procedure is not universally applicable, in particular it cannot be used when the polyol used for the NCO prepolymer does not have solely or at least predominantly primary OH groups. The person skilled in the art knows that, in particular when using cycloaliphatic diisocyanates, such as, for example, isophorone diisocyanate, with polyether polyols that do not have solely or predominantly primary OH groups, such teaching results in economically unacceptably long reaction times for the prepolymer preparation. The same is also true of the reaction of such NCO prepolymers with amine-terminated aminoalkylalkoxysilane. Even with dibutyltin dilaurate catalysis, for example, phases which are of long duration and therefore uneconomical are passed through, during which free amine is present in addition to free isocyanate. For dental applications, the more reactive aromatic polyisocyanates are excluded from the outset because of their toxicity. Free isocyanate, whether it be of aromatic or aliphatic nature, is, of course, fundamentally no more tolerable in dental applications than an excess of aminosiloxane that exceeds an absolute minimum.

In addition, free isocyanates are also not acceptable because they would slowly react further over time, for example after compounding with additives and auxiliary substances, as a result of which the consistency of the paste slowly changes and the storage stability could accordingly not be ensured.

Some of the last-mentioned aspects are already described in EP-A 0 269 819. However, EP-A 0 269 819 does not describe whether, and where appropriate, which type of catalysts are advantageously to be used for the complete reaction of the NCO groups. Only tin octoate is used in two examples.

However, tin compounds lead to the problem of corrosion effects when they are stored in particular packing agents such as aluminium tubes or aluminium-based pouches. In addition, toxicological objections to organotin compounds have increasingly been expressed recently. There is therefore a need for dental molding compositions which preferably do not contain tin compounds, but in which the content of tin compounds is at least limited to a minimum, for example 5 ppm, that is to say an order of magnitude of about 10% of the amount that is conventional. A solution to this problem cannot be found in the teaching of EP-A 0 269 819.

The same is true of EP-A 0 096 249, EP-A 0 158 893, U.S. Pat. No. 4,374,237 and U.S. Pat. No. 3,632,557, DE-A 4 307 024, EP-A 0 687 280, DE-A 4439 769, DE-A 10 201 703, EP-A 1 563 822, EP-A 1 563 823 as well as EP-A 1 226 808, EP-A 1 402 873 and EP-A 1 081 191.

Furthermore, EP-A 0 269 819 teaches that preference is given to the use of polyethers that contain predominantly, that is to say up to 90%, primary OH end groups, based on all OH end groups. The only economically relevant polyether polyols, apart from the polytetrahydrofurans, are those prepared from ethylene and/or propylene oxide. Polytetrahydrofurans are less suitable for dental applications because they exhibit a phase transition in the region of room temperature, with the result that the flow properties, and accordingly the processing properties, are dependent on temperature to an undesirably great extent in the region of the use temperature. A further disadvantage is their high cost compared with types based on ethylene/propylene oxide. In the case of ethylene/propylene-oxide-based polyethers, high primary OH group contents are, of course, only obtained by polymerizing relatively large amounts of ethylene oxide units, optionally in admixture with propylene oxide, onto polypropylene oxide as the terminal block during the preparation of such polyethers. That structure in turn leads to an undesirably high degree of hydrophilicity, which has a strongly negative effect on the water absorption behavior and accordingly on the storage stability of the pastes prepared therewith. It is therefore desirable in this connection to be able to use polyethers having as few ethylene oxide structural elements as possible and nevertheless ensure acceptable reaction times.

The object underlying the present invention was, therefore, to provide an impression composition system based on silane-terminated polyether derivatives for the dental sector, which system does not contain tin compounds, if possible, or has a maximum content of tin compounds of <5 ppm, which impression system must be capable of being produced economically and must fulfill all the demands made of dental impression compositions mentioned at the beginning.

Surprisingly, it has now been found that this object can be achieved in an outstanding manner by means of silane-terminated polyethers which are prepared substantially or wholly without catalysis by tin compounds.

### SUMMARY OF THE INVENTION

The invention accordingly provides silane-terminated polyether derivatives, obtainable by
1) preparing a prepolymer by reacting
a.) one or more largely linear polyether polyols having predominantly secondary OH groups, with
b.) one or more diisocyanates
   wherein the prepolymer-forming reaction is catalyzed by a catalyst or catalyst mixture having not more than 5 ppm tin, based on the weight of said prepolymer, and said prepolymer having a NCO content of from 0.5 to 6 wt. % NCO, preferably from 1 to 4 wt. % NCO, and
2) reacting the prepolymer in a second reaction step with
c.) one or more amino-group-containing compounds of the general formula (i)

HNR—(CH₂)ₙ—SiR₁R₂R₃ (i)
wherein
R represents hydrogen or —(CH₂)ₙ—SiR₁R₂R₃,
n represents an integer from 1 to 6, and
at least one of the groups R₁, R₂, R₃ has the structure (—O—CₚH₂ₚ)_{q}—OR₄,
wherein
p has values from 2 to 5, preferably 3, and
q has values from 0 to 100, preferably from 0 to 4, and
R₄ represents a substituent selected from the group comprising alkyl, aryl, arylalkyl, vinyl and vinylcarbonyl
and
the remaining groups R₁, R₂, R₃ are alkoxy radicals having from 1 to 4 carbon atoms,
   in such a manner that the NCO value is less than 0.001 wt. % NCO and the content of free amino groups is in the range from 0.5 to 50 mmol, preferably from 1 to 15 mmol, particularly preferably from 0.5 to 5 mmol of amine groups per kg of the silane-terminated polyether derivative so obtained.

The invention further provides a process for the preparation of silane-terminated polyether derivatives, comprising

1) preparing a prepolymer by reacting
a.) one or more largely linear polyether polyols having predominantly secondary OH groups, are reacted, with the aid of catalysts, with
b.) one or more diisocyanates
   wherein the prepolymer-forming reaction is catalyzed by a catalyst or catalyst mixture having not more than 5 ppm tin, based on the weight of said prepolymer, and said prepolymer having a NCO content of from 0.5 to 6 wt. % NCO, preferably from 1 to 4 wt. % NCO,
   2) reacting the prepolymer with
c.) one or more amino-group-containing compounds of the general formula (i)

HNR—(CH₂)ₙ—SiR₁R₂R₃ (i)
wherein
R represents hydrogen or —(CH₂)ₙ—SiR₁R₂R₃,
n represents an integer from 1 to 6, and
at least one of the groups R₁, R₂, R₃ has the structure (—O—CₚH₂ₚ)_{q}—OR₄,
wherein
p has a value from 2 to 5, preferably 3, and
q has a value from 0 to 100, preferably from 0 to 4, and
R₄ represents a substituent selected from the group consisting of alkyl, aryl, arylalkyl, vinyl and vinylcarbonyl
and
the remaining groups R₁, R₂, R₃ are alkoxy radicals having from 1 to 4 carbon atoms, and
   3) optionally reacting the product of step 2) with an aliphatic isocyanate,
   in such a manner that the NCO value is less than 0.001 wt. % NCO and the content of free amino groups is in the range from 0.5 to 50 mmol, preferably from 1 to 15 mmol, particularly preferably from 0.5 to 5 mmol of amino groups per kg of the silane-terminated polyether derivative so obtained.

### DETAILED DESCRIPTION OF THE PREFERRED EMBODIMENTS

The invention is described in greater detail below.

As used herein and in the claims, the phrase “largely linear” shall mean having a hydroxyl functionality from 1.95 to 2.3, preferably from 1.96 to 2.06.

As used herein and in the claims, the phrase “predominantly secondary OH groups” shall mean at least 80% of the OH groups are secondary OH groups.

In accordance with the process according to the invention for the preparation of silane-terminated polyether derivatives, largely linear polyether polyols having at least 80% secondary OH groups are reacted in a first reaction step, with the aid of zinc catalysts, by reaction with aliphatic polyisocyanates to form a prepolymer having a NCO content of from 0.5 to 6 wt. % NCO, preferably from 1 to 4 wt. % NCO.

Largely linear polyether polyols having more than 80% secondary OH groups are those polyols which are obtained by ring-opening polymerization from epoxides, for example ethylene and propylene oxide, preferably wholly or predominantly propylene oxide, with the aid of, for example, KOH or double metal catalysts (DMCs) as catalysts, using starter compounds containing reactive hydrogen atoms from the group of the polyalcohols and polyamines, and water. Preference is given to divalent starter compounds, such as, for example, ethylene glycol, 1,2-propylene glycol, 1,3-propylene glycol, diethylene glycol, 1,4-butylene glycol, 2,3-butylene glycol, 1,6-hexanediol, glycerol, 1,1,1-trimethylolpropane and water. Starter compounds according to the invention also include mixtures of a plurality of starter compounds, the composition of the starter mixtures being such that polyether polyols having an OH functionality of not more than 2.5, preferably not more than 2.2, are formed.

If more than one epoxide is used, then the polymerization can take place either block-wise or mixed. It is preferred, however, to use only one epoxide, particularly preferably propylene oxide, as well as mixtures of two epoxides, the mixtures consisting predominantly of propylene oxide.

Polyether polyols according to the invention are further characterized in that they have number-average molecular weights of from 150 to 20,000 Da, preferably from 500 to 6500 Da, particularly preferably from 800 to 5500 Da. Of course, mixtures of at least two polyether polyols can advantageously also be used, in which case the number-average molecular weight of the mixture is within the range described above.

Examples of aliphatic polyisocyanates are 4,4′-methylenebis(cyclohexyl isocyanate), ethylene diisocyanate, tetramethylene diisocyanate, hexamethylene diisocyanate, dodecamethylene diisocyanate, cyclobutane-1,3-diisocyanate, cyclohexane-1,3-diisocyanate, cyclohexane-1,4-diisocyanate or 1-isocyanato-3,3,5-trimethyl-5-isocyanatomethyl-cyclohexane (isophorone diisocyanate, IPDI). They can be used individually or in a mixture, but IPDI is particularly preferred.

In a first reaction stage, the polyethers according to the invention are reacted with polyisocyanates according to the invention, in accordance with the prior art, at temperatures in the range from 60 to 150° C., preferably from 80 to 110° C., preferably using a protecting gas, particularly preferably nitrogen, at normal pressure to reduced pressure, preferably normal pressure, to form NCO prepolymers, it being possible to use a solvent that is inert towards NCO groups, it being preferred, however, to work without a solvent.

In order to accelerate the reaction, catalysts are used in accordance with the invention. Preferred catalysts, optionally catalyst mixtures, are characterized in that the silane-terminated polyether derivatives contain maximum amounts of 5 ppm tin compound. Preference is given to the use of catalysts that contain wholly or predominantly zinc as the metal atom. Examples of catalysts according to the invention are zinc acetate, zinc citrate, zinc lactate, zinc stearate, zinc undecylenate, preferably zinc di-tert.-butyl salicylate, zinc acetylacetonate and zinc neodecanoate. The catalysts are advantageously used in amounts of from 0.5 to 10 mg of Zn/kg of prepolymer.

The prepolymers according to the invention have NCO contents of from 0.5 to 6 wt. % NCO, preferably from 1 to 4 wt. % NCO.

The prepolymer formation is regarded as being complete when the NCO content determined in practice reaches the theoretically calculated NCO value.

The NCO prepolymers according to the invention are then reacted with alkoxysilylmonoamines in a second reaction stage. Suitable alkoxysilylmonoamines are known. Examples include the γ-aminopropyl-tri-C₁-C₄-alkoxysilanes or bis-(3-C₁-C₄-alkoxysilylpropyl)amines which are readily available commercially, such as, for example, γ-aminopropyltrimethoxysilane and γ-aminopropyltriethoxysilane.

The reaction of NCO prepolymer and alkoxysilylmonoamine to yield reactive silane-terminated polyether derivatives is so carried out that no more NCO can be detected in the silane-terminated polyether derivative and the content of free amino groups is in the range from 0.5 to 50 mmol, preferably from 1 to 15 mmol, particularly preferably from 0.5 to 5 mmol of amine groups per kg of silane-terminated polyether derivatives.

Those specifications are preferably achieved according to the invention by first stirring in, at elevated temperature, preferably at least 50° C., a stoichiometric excess of alkoxysilylmonoamine which is mathematically suitable for adjusting the NCO value to 0 and the amine value to a value of preferably from 0.5 to 5 mmol per kg of silane-terminated polyether derivative, and allowing the mixture to react. At this stage of the reaction, both free amine and free isocyanate are found. After about 2 hours, the amine content and the NCO content are determined hourly. The reaction is regarded as being complete when one of the values of two successive measurements is unchanged. If the amine value is within the desired range and at the same time the NCO value is 0, the product is finished. If the amine value is zero and the NCO value is >0, an amount of alkoxysilylmonoamine sufficient to raise the amine value to a range of from 0.5 to 5 mmol of amine groups per kg is metered in.

If the amine value is above the desired range and the NCO value is zero, an amount of aliphatic monoisocyanate that is mathematically sufficient to lower the amine value to the desired range is metered in.

The use of the aliphatic monoisocyanate instead of (alternatively) IPDI with at least one very slow reacting NCO group represents a substantial advantage in terms of time.

In a further preferred variant according to the invention, the condition of a silane-terminated polyether derivative having a NCO value of zero and an amine value in the range from 0.5 to 5 mmol of amine groups per kg of silane-terminated polyether derivative is achieved by first adding a more than stoichiometric amount of alkoxysilylmonoamine and, optionally by subsequently metering in the same compound, adjusting the amine group content to constant values greater than 2 mmol of amine groups per kg of polyurethane composition, particularly preferably from 2 to 5 mmol of amine groups per kg of polyurethane composition, and by reducing that value above 2 mmol to values below 2 mmol/kg by addition of a less than stoichiometric amount, based on the amine groups, of an aliphatic isocyanate, preferably a monoisocyanate having at least 2 carbon atoms, preferably at least 6 carbon atoms, such as, for example, 1-n-octyl isocyanate, 1-n-decyl isocyanate, 1-n-dodecyl isocyanate or 1-stearyl isocyanate, relative to the amount of free amino groups.

Of course, it is possible by means of the process according to the invention also to establish conditions other than the above-mentioned status in respect of NCO value and amine group concentration.

The molding compositions according to the invention based on silane-terminated polyether derivatives are provided with further auxiliary substances and additives, in accordance with the prior art, in order to bring them into a form capable of application.

Examples which may be mentioned include: fillers, colorings, pigments, thickeners, surfactants, fragrances and flavorings, and also diluents.

Water is required for the curing reaction in the oral cavity. In order to establish practicable curing times, acids are added as catalytically active components. Dental molding compositions according to the invention are preferably supplied in the form of two-component systems, one component containing the silane-terminated polyether derivatives and optionally further auxiliary substances and additives, and the other component containing water, one or more acidic components and optionally auxiliary substances and additives.

It is surprising that
the described silane-terminated polyether derivatives prepared with Zn catalysis have a molecular weight distribution comparable to that of silane-terminated polyether derivatives prepared with catalysis by means of tin compounds and having contents of tin compounds >5 ppm;
the systems according to the invention have comparable or more advantageous storage stability;
the molding compositions obtained with the silane-terminated polyether derivatives used according to the invention fulfill the fundamental demands made of molding materials and do not differ substantially in terms of their physical and application-related property profile from the compositions according to the prior art having contents of tin compounds >5 ppm.

The examples which follow explain the invention further and illustrate the technical effects associated therewith.

### EXAMPLES

### Example 1 (in Accordance with the Invention)

### Preparation of the Polyurethane Compositions

2553 g of a polypropylene oxide having an OH number of 28 mg KOH/g (Acclaim® 4200N (Bayer MaterialScience AG)) which had previously been dewatered under a water-jet vacuum were heated to 100° C., and 236 g of isophorone diisocyanate (IPDI) were added thereto in the course of 2 minutes, with stirring, under a protecting gas. After 5 minutes, 100 mg of zinc di-tert.-butyl salicylate were added. Stirring was carried out for about 2 hours at 100° C. and the NCO content of the NCO prepolymer was determined as 1.20 wt. % NCO (theoret.: 1.28 wt. %).

The mixture was allowed to cool to 40° C. and the NCO content was determined again (1.20 wt. % NCO).

170 g of Dynasilan® Ameo (adhesive TP 3023, Degussa AG) were stirred into the viscous reaction mass at 40° C. After 2 hours and 3 hours, the content of free amine was determined as 0.5 mmol of amine/kg.

A further 1 g of Dynasilan® Ameo was stirred in, the amine content being determined after 2 hours and after 3 hours as 0.4 mmol of amine/kg.

A further 2 g of Dynasilan® Ameo were stirred in, the amine content being determined after 2 and 3 hours as 0.3 mmol of amine/kg.

A further 4 g of Dynasilan® Ameo were stirred in, the amine content being determined after 2 and 3 hours as 1.8 mmol of amine/kg.

The increase in the content of free amine after the last addition of Dynasilan® Ameo indicates that all the NCO groups have reacted completely.

The NCO value at that time was determined as 0 wt. % NCO. The amine content determined after a further 24 hours was constant at 1.8 mmol of amine/kg.

### Example 2 (in Accordance with the Invention)

### Preparation of the Polyurethane Compositions

2550 g of a polypropylene oxide having an OH number of 28 mg KOH/g (Acclaim® 4200N (Bayer MaterialScience AG)) which had previously been dewatered under a water-jet vacuum were heated to 100° C., and 283 g of isophorone diisocyanate (IPDI) were added thereto in the course of 2 minutes, with stirring, under a protecting gas. After 5 minutes, 80 mg of zinc di-tert.-butyl salicylate were added. Stirring was carried out for about 2 hours at 100° C. and the NCO content of the NCO prepolymer was determined as 1.83 wt. % NCO (theoret.: 1.89 wt. %).

The mixture was allowed to cool to 40° C. and the NCO content was determined again (1.83 wt. % NCO).

273 g of Dynasilan® Ameo were stirred into the viscous reaction mass at 40°.

After 2 hours and 3 hours, the content of free amine was determined as 1.99 mmol of amine/kg.

A further determination of the content of free amine after 24 hours gave a content of free amine of 1.98 mmol of amine/kg. The NCO value at that time was determined as 0 wt. % NCO.

### Comparison Example 1

### CE1, Not in Accordance with the Invention

The same procedure as in Example 1 was used, but 150 mg of dibutyltin dilaurate were added as the catalyst instead of Zn tert.-butyl salicylate.

After stirring for 2 hours at 100° C., the NCO content of the NCO prepolymer was determined as 1.25 wt. % NCO (theoret.: 1.28 wt. %).

The mixture was allowed to cool to 40° C. and the NCO content was determined again (1.25 wt. % NCO).

180 g of Dynasilan® Ameo were stirred into the viscous reaction mass at 40° C. After 2 hours and 3 hours, the content of free amine was determined as 0.11 mmol of amine/kg.

A further 2.7 g of Dynasilan® Ameo were stirred in, the amine content being determined after 2 hours and after 3 hours as 2.96 mmol of amine/kg.

0.45 g of octyl isocyanate was stirred in, the amine content being determined after 2 and 3 hours as 1.74 mmol of amine/kg.

The NCO value at that time was determined as 0 wt. % NCO. The amine content determined after a further 24 hours was constant at 1.74 mmol of amine/kg.

### Comparison Example 2

### CE2, Not in Accordance with the Invention

The same procedure as in Example 1 was used, but 150 mg of dibutyltin dilaurate were added as the catalyst instead of Zn tert.-butyl salicylate.

After stirring for 2 hours at 100° C., the NCO content of the NCO prepolymer was determined as 1.82 wt. % NCO (theoret.: 1.89 wt. %).

The mixture was allowed to cool to 40° C. and the NCO content was determined again (1.82 wt. % NCO).

269 g of Dynasilan® Ameo were stirred into the viscous reaction mass at 40° C. After 2 hours and 3 hours, the content of free amine was determined as 0.3 mmol of amine/kg.

A further 1 g of Dynasilan® Ameo was stirred in, the amine content being determined after 2 hours and after 3 hours as 1.52 mmol of amine/kg.

The NCO value at that time was determined as 0 wt. % NCO.

In order to determine the molecular weight distribution, tests by means of gel permeation chromatography were carried out. It was clear from these tests that the molecular weight distributions of E1 and CE1 and of E2 and CE2 largely correspond.

### Test of Storage Stability

The products from Examples 1, 2 and Comparison Examples CE1 and CE2 were packed in an air-tight manner and stored at 60° C. In order to assess the storage stability, the change in viscosity was determined.

The silane-terminated polyether derivatives used according to the invention are distinguished by a similar or smaller change in viscosity and accordingly by comparable or greater storage stability:

TABLE 1
Determination of the storage stability of silane-terminated polyether derivatives

| | | | | Viscosity | Viscosity |
|---|---|---|---|---|---|
| | | | Viscosity | (23° C., 3 s⁻¹) | (23° C., 3 s⁻¹) |
| | | | (23° C., 3 s⁻¹) | after | after |
| Silane- | Content of | Content of | immediately | 1 month's | 2 months' |
| terminated | tin | zinc | after | storage at | storage at |
| polyether | compound | compound | preparation | 60° C. | 60° C. |
| derivative | [ppm] | [ppm] | [Pas] | [Pas] | [Pas] |
| acc. to Ex. 1 | 0 | 33 | 127 | 148 | 167 |
| acc. to Ex. 2 | 0 | 26 | 103 | 122 | 115 |
| acc. to CE1 | 50 | 0 | 126 | 161 | 185 |
| acc. to CE2 | 50 | 0 | 100 | 131 | 146 |

Table 1 shows that it is possible according to the invention to obtain systems whose storage stabilities are at least equal to, and on prolonged storage superior to, those of conventionally catalyzed systems.

### Formulation Examples

### A. Preparation of the Base Components

In a laboratory dissolver, 20 parts by weight of the silane-terminated polyether derivatives were mixed for 3 hours at a pressure <50 mbar with 20 parts by weight of dibenzyltoluene, 56 parts by weight of quartz powder and 4 parts by weight of hydrogenated castor oil to give a homogeneous pasty mass.

### B. Preparation of the Catalyst Component is Carried Out According to DE-A 10 104 079

### Example 3

The various base components were mixed with the catalyst component in a weight ratio of 5:1 in each case. The processing time (in accordance with DIN EN ISO 4823), the Shore A hardness (in accordance with DIN 5305) and the resistance to tearing (in accordance with DIN 53504) of the blends were determined. The compositions according to the invention corresponded in each case with the property profile of the compounds prepared with tin catalysis. The tin-free molding compositions according to the invention fulfill the fundamental demands made of dental impression compositions (according to ISO 4823).

TABLE 2
Formulations for the preparation of dental impression
compositions and testing of important properties

| | | in accordance | Comparison example, |
|---|---|---|---|
| | | with the | not in accordance |
| | | invention | with the invention |
| Formulation: | | | |
| E1 | [parts] | 10 | |
| E2 | [parts] | 10 | |
| CE1 | [parts] | | 10 |
| CE2 | [parts] | | 10 |
| Dibenzyltoluene | [parts] | 20 | 20 |
| Quartz powder | [parts] | 56 | 56 |
| Hydrogenated | [parts] | 4 | 4 |
| castor oil | | | |
| Content of tin | [ppm] | <2 | 10 |
| compound | | | |
| Content of zinc | [ppm] | 6 | <2 |
| compound | | | |
| Properties: | | | |
| Processing time | [min] | 1.8 | 1.8 |
| Curing time | [min] | | |
| Recovery after | [%] | 98.5 | 98.6 |
| deformation | | | |
| Deformation | [%] | 4.0 | 4.1 |
| under pressure | | | |
| Shore A (1 hour) | [Shore A] | 61 | 57 |
| Resistance to | [MPa] | 2.9 | 2.6 |
| tearing | | | |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A silane-terminated polyether derivative, obtained by
1) preparing a prepolymer by reacting:
a.) one or more largely linear polyether polyols having predominantly secondary OH groups, with
b.) one or more diisocyanates;
wherein the prepolymer-forming reaction is catalyzed by a catalyst or catalyst mixture having not more than 5 ppm tin, based on the weight of said prepolymer, and said prepolymer having a NCO content of from 0.5 to 6 wt. % NCO, and
2) reacting the prepolymer with
c.) one or more amino-group-containing compounds of the general formula (i)
HNR—(CH₂)ₙ—SiR₁R₂R₃ (i)
wherein
R represents hydrogen or —(CH₂)ₙ—SiR₁R₂R₃,
n represents an integer from 1 to 6, and
at least one of the groups R₁, R₂, R₃ has the structure (—O—CₚH₂ₚ)_{q}—OR₄,
wherein
p has a value of from 2 to 5, and
q has a value from 0 to 100, and
R₄ represents a substituent selected from the group consisting of alkyl, aryl, arylalkyl, vinyl and vinylcarbonyl,
and
the remaining groups R₁, R₂, R₃ are alkoxy radicals having from 1 to 4 carbon atoms,
in amounts such that the silane-terminated polyether derivative has an NCO value of less than 0.001 wt. % NCO and the content of free amino groups is in the range from 0.5 to 50 mmol of amine groups per kg of silane-terminated polyether derivative.

2. A silane-terminated polyether derivative according to claim 1, wherein the content of free amino groups is in the range from 1 to 15 mmol of amine groups per kg of the silane-terminated polyether derivative.

3. A silane-terminated polyether derivative according to claim 1, wherein the content of free amino groups is in the range from 0.5 to 5 mmol of amine groups per kg of the silane-terminated polyether derivative.

4. A process for the preparation of silane-terminated polyether derivatives, comprising
1) preparing a prepolymer by reacting
a.) one or more largely linear polyether polyols having predominantly secondary OH groups are reacted, with
b.) one or more diisocyanates
wherein the prepolymer-forming reaction is catalyzed by a catalyst or catalyst mixture having not more than 5 ppm tin, based on the weight of said prepolymer, and said prepolymer having a NCO content of from 0.5 to 6 wt. % NCO,
2) reacting the prepolymer with
c.) one or more amino-group-containing compounds of the general formula (i)
HNR—(CH₂)ₙ—SiR₁R₂R₃ (i)
wherein
R represents hydrogen or —(CH₂)ᵣ—SiR₁R₂R₃,
n represents an integer from 1 to 6, and
at least one of the groups R₁, R₂, R₃ has the structure (—O—CₚH₂ₚ)_{q}—OR₄,
wherein
p has a value from 2 to 5, and
q has a value from 0 to 100, and
R₄ represents a substituent selected from the group consisting of alkyl, aryl, arylalkyl, vinyl and vinylcarbonyl
and
the remaining groups R₁, R₂, R₃ are alkoxy radicals having from 1 to 4 carbon atoms, and
3) optionally reacting the product of step 2) with an aliphatic isocyanate and/or one or more compounds according to c.),
in amounts such that the silane-terminated polyether derivative has an NCO value of less than 0.001 wt. % NCO and the content of free amino groups is in the range from 0.5 to 50 mmol, of amino groups per kg of silane-terminated polyether derivative.

5. A process according to claim 4, wherein the catalyst or catalyst mixture comprises at least one further catalytically active species other than tin.

6. A process according to claim 5, wherein the catalyst or catalyst mixture comprises one or more zinc salts.

7. A process according to claim 6, wherein the one or more zinc salts are selected from the group consisting of zinc di-tert.-butyl salicylate, zinc acetylacetonate and zinc neodecanoate.

8. A process according to claim 4, wherein the catalyst or catalyst mixture comprises from 0.5 to 10 mg of Zn/kg of prepolymer.

9. A process according to claim 4, wherein the prepolymer-forming reaction occurs at temperatures of from 60 to 150° C., under protecting gas,

10. A process according to claim 4, wherein the aliphatic isocyanate is selected from the group consisting of 1-n-octyl isocyanate, 1-n-decyl isocyanate, 1-n-dodecyl isocyanate and 1-stearyl isocyanate.

11. A molding composition comprising the silane-terminated polyether derivative of claim 1.

12. The molding composition of claim 11, wherein the composition is suitable for dental applications.

13. The molding composition of claim 12, wherein the composition is a two-component system comprising a first component containing the silane-terminated polyether of claim 1 and optionally further auxiliary substances and additives, and a second component containing water, one or more acidic compounds and optionally auxiliary substances and additives.
